(19) United States
(12) Patent Application Publication
(10) Pub. No.: US 2026/0321384 A1
(43) Pub. Date: Oct. 15, 2026

(54) ILLUMINATION MODULE FOR MEDICAL FLUID INFUSION PUMPS

(71) Applicant: CHS HEALTHCARE VENTURES, INC., Decatur, GA (US)

(72) Inventors: Hans Utz, Decatur, GA (US); Marshall Fryman, Decatur, GA (US); Dragan Nebrigic, Austin, TX (US)

(73) Assignee: CHS HEALTHCARE VENTURES, INC, Decatur, GA (US)

(21) Appl. No.: 19/677,826

(22) Filed: May 14, 2026

Related U.S. Application Data
(63) Continuation-in-part of application No. 19/453,516, filed on Jan. 20, 2026.
Application No. 19/453,516 is a continuation of application No. 17/578,707, filed on Jan. 19, 2022, now Pat. No. 12,555,661.
(60) Provisional application No. 63/138,961, filed on Jan. 19, 2021.
(60) Provisional application No. 63/805,747, filed on May 14, 2025.

(51) Int. Cl. G16H 20/17; A61L 103/15; A61M 5/14; A61M 5/145; A61M 5/158; A61M 5/162; A61M 5/172; F21V 33/00; G02B 6/44; G16H 40/63; H05B 45/10; H05B 47/11

(52) U.S. Cl. CPC G16H 20/17; A61M 5/14; A61M 5/1582; A61M 5/162; A61M 5/172; F21V 33/0068; G02B 6/4442; G02B 6/4482; G16H 40/63; H05B 45/10; H05B 47/11; A61L 2103/15; A61L 2202/14; A61M 2005/1401; A61M 2005/14553; A61M 2205/14; A61M 2205/3306; A61M 2205/3313; A61M 2205/3317; A61M 2205/3633; A61M 2205/587; A61M 2205/6054; A61M 2205/6081; A61M 2205/8206; A61M 2207/00; F21V 2200/10

(57) ABSTRACT

Aspects of systems and apparatuses for a medical infusion illuminator within a protective housing are disclosed. The systems and apparatuses include an electronic illuminator module attached to a medical infusion pump. The module comprises a protective housing containing a printed circuit board (PCB), a light emitting diode (LED), a lens, and a power source electrically connected to the medical infusion pump, along with at least one sensor assembly. A side scattering fiber optic cable is connected to the electronic illuminator module, featuring a fiber funnel cap at its proximal end to receive light from the LED through the lens. The distal end of the fiber optic cable is equipped with a protective end cap, polished on its interior surface to reflect light from the LED, ensuring effective illumination along the infusion line. This system enhances visibility and monitoring of medical infusion lines, improving safety and efficiency in medical settings.

### CROSS-REFERENCE TO RELATED APPLICATIONS

This application claims the benefit of and priority to U.S. Provisional Application 63/805,747 entitled Illumination Module for Medical Fluid Infusion Pumps filed on May 14, 2025; This application is further a continuation-in-part of and claims the benefit and priority to U.S. patent application Ser. No. 19/453,516 entitled Electronic Illuminator for Illuminating Medical Infusion Lines, filed on Jan. 1, 2026, which further claims the benefit of and priority to U.S. Pat. No. 12,555,661, entitled Medical Infusion Line Electronic Illuminator, which further claims the benefit of and priority to U.S. Provisional Application 63/138,961, filed on Jul. 21, 2022.

### FIELD

The present invention relates to electronic illumination of side emitting fiber optic lines with an electronic illumination module. In particular, a system and method for providing an illumination module coupled to a medical infusion pump to provide illumination of side emitting fiber optic lines for medical infusion line intelligence.

### BACKGROUND

The present disclosure relates generally to systems, apparatuses, and methods for improving the administration of medical infusion utilizing advanced sensor assemblies and leveraging computational intelligence to improve patient care and reduce practitioner cognitive load. Medical infusion serves to administer medications, fluids, nutrients, solutions, and other materials intravenously to a patient. Patients are often administered medical infusion using intravenous infusion tubing or lines (‘IVT’). Such intravenous infusion tubing generally consists of flexible, polymer tubing connected at one end to a fluid source and at another end to a needle or port assembly that provides access to a blood vessel of a patient. It is not uncommon for many infusion tubes, each connected to a different source of fluid (medical infusion pump), to be used simultaneously to deliver several medications at once to a single patient. It is also not uncommon for the needles or port assemblies to be located adjacent to one another, such as multiple adjacent needles, providing access into the brachial vein running through the arm of the patient.

Distinguishing between multiple infusion tubes is a difficult task, and medication delivery error as a result of improperly distinguishing one infusion tube from another is a serious problem in current infusion systems. The confusion of one infusion tube from another is one of the leading causes of preventable medication error. It is potentially life-threatening and is a serious and ongoing concern and cost to medical facilities.

Distinguishing between multiple infusion tubes is a difficult task that is placed in an atmosphere of high stress and rapid timing. The medical industry refers to the atmosphere as placing a high cognitive load on practitioners. This high cognitive load can lead to medication delivery error as a result of improperly distinguishing one medical infusion tube from another. Arguably, the confusion of one medical infusion tube from another is one of the leading causes of preventable medication error. As a result of the difficulties in distinguishing between multiple medical infusion tubes and their associated fluid sources and outputs, as well as the potentially life-threatening possibilities that can occur if incompatible medications are injected through the same medical infusion line; therefore, there is a need for accurate identification of medical infusion tubes. The problem is addressed with an array of sensors, assemblies, processors, physics, and utilizing electrical and computational engineering to improve upon and develop advanced systems of an electronic illuminator and side emitting fiber optics to facilitate illumination, detection, and verification of medical infusion tubes. Further, the systems and methods herein are engineered to improve the usage of an electronic illuminator by providing feedback and reliability into the operation thereof.

### SUMMARY

Aspects of systems and apparatuses for medical infusion illumination through a module are disclosed herein. In some aspects, the techniques described herein relate to a system for medical infusion line illumination, including: (i) an electronic illuminator module attached to a medical infusion pump, the module including: a protective housing, inside the protective housing is a printed circuit board (‘PCB’), a light emitting diode (‘LED’), a lens, and a power source, wherein the power source is in electrical communication to the medical infusion pump; and at least one sensor assembly. (ii) a side scattering fiber optic cable connected to the electronic illuminator module, the side scattering fiber optic cable including: a fiber funnel cap at a proximal end to receive light from the LED through the lens of the electronic illuminator module; and a protective end cap at a distal end, wherein the protective end cap is polished on an interior surface to reflect light from the LED of the electronic illuminator module.

### BRIEF DESCRIPTION OF THE DRAWINGS

Many aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure. Therefore, in the drawings:

FIG. 1A is perspective view of an illustration of an example electronic illuminator, displaying the configuration of internal components and subsystems;

FIG. 1B is an exploded view of an illustration of an example electronic illuminator;

FIG. 2 is a schematic diagram of an example of an electronic illuminator's internal components and subsystems;

FIG. 3 is a schematic diagram of an example of an electronic illuminator's LED power system drivers;

FIG. 4 is a block diagram of an example of an electronic illuminator's ambient light sensor;

FIG. 5 is a schematic diagram of an example of an electronic illuminator's ambient light sensor chipset;

FIG. 6 is an illustration of an example of an electronic illuminator in cross section, disclosing the ambient light sensor configuration within the electronic illuminator;

FIG. 7 is an illustration of an example of an electronic illuminator's Rigid-Flex PCB, wherein the flex portion is disclosed with the ambient light sensor and the cap color detection assembly;

FIG. 8 is a block diagram of an example of an electronic illuminator's cap color detection assembly;

FIG. 9 is a schematic diagram of an example of an electronic illuminator's R/G/B sensor forming a part of the cap color detection assembly;

FIG. 10 is an illustration of an example of an electronic illuminator's Rigid-Flex PCB, wherein the flex portion is disclosed with the cap color detection assembly;

FIG. 11 is an illustration of an example of an electronic illuminator, disclosing the cap color detection assembly configuration within the electronic illuminator;

FIG. 12 is an illustration of an example cap and magnet array for an electronic illuminator, the configuration provides aspects of the fiber detection assembly;

FIG. 13 is an additional illustration of an example cap and magnet array with a steel bar for an electronic illuminator, the configuration provides aspects of the fiber detection assembly;

FIG. 14 is an illustration of an example fiber detection assembly, wherein the Rigid-Flex PCB is disclosed with a Hall sensor;

FIG. 15 is an illustration of an example distal end cap, also known as a protective end cap on a side emitting fiber optic line;

FIGS. 16A-C are illustrations of an example fiber funnel cap that is designed to operatively engage with an electronic illuminator or position itself near the lens and LED to transmit light along the side scattering fiber optic line.

FIG. 17 is a perspective view of an example medical infusion pump with an electronic illuminator module attached to the bottom and the corresponding side scattering fiber optic line attached to the medical fluid line of the medical infusion pump;

FIG. 18A is a perspective view of an example electronic illuminator module; and

FIG. 18B is a diagram of an example electronic illuminator module with the housing removed.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Throughout this specification and the claims, the terms “fiber optic cable,” “fiber optic line,” and “fiber optic” are used to mean a side scattering or side emitting or side glow fiber optic cable, wherein light or illumination is purposefully emitted as it traverses the length of the cable or line.

Throughout this specification and the claims, the terms “comprise,” “comprises,” and “comprising” are used in a non-exclusive sense, except where the context requires otherwise. Likewise, the term “includes” and its grammatical variants are intended to be non-limiting, such that recitation of items in a list is not to the exclusion of other like items that can be substituted or added to the listed items.

### I. Example Use Case Scenarios

Medical infusion typically serves to administer medications, fluids, nutrients, solutions, and other materials intravenously to a patient. Patients are often administered medical infusion using intravenous infusion lines. Such intravenous infusion lines generally consist of flexible, plastic tubing connected at one end to a fluid source and at another end to a needle or port that provides access to a blood vessel of a patient. It is not uncommon for many infusion lines, each connected to a different source of fluid (medical infusion pump), to be used simultaneously to deliver several medications at once to a single patient. It is also not uncommon for the needles or ports to be located adjacent one another, such as multiple adjacent needles providing access into the brachial vein running through the arm of the patient.

Distinguishing between multiple infusion lines is a difficult task that is placed in an atmosphere of high stress and rapid timing. The medical industry refers to the atmosphere as placing a high cognitive load on practitioners. This high cognitive load can lead to medication delivery error as a result of improperly distinguishing one medical infusion line from another. Arguably, the confusion of one medical infusion line from another is one of the leading causes of preventable medication error. As a result of the difficulties in distinguishing between multiple medical infusion lines and their associated fluid sources and outputs, as well as the potentially life-threatening possibilities that can occur if incompatible medications are injected through the same medical infusion line, there is a need for accurate identification of medical infusion lines.

Similarly, the disclosure herein may also be applied to many other environments where a cognitive load, or otherwise environment restrictions, may place a burden on traditional techniques. For example, tracing of lines within the Information Technology (‘IT’) field may enable rapid detection and identification. In other aspects, the disclosed embodiments may be useful for the energy sector, where illumination needs may be utilized to send signals through long distances and to have a system that may rapidly deploy and integrate with little additional cost. Therefore, there is a need to improve electronic illuminators with a more advanced sensor system, and provide on board intelligence that allows multi-functional use and adaptability.

### II. Systems and Methods

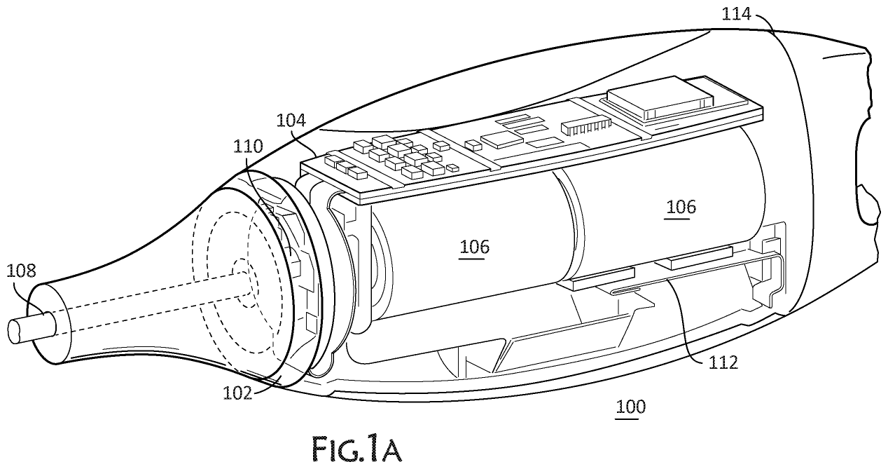

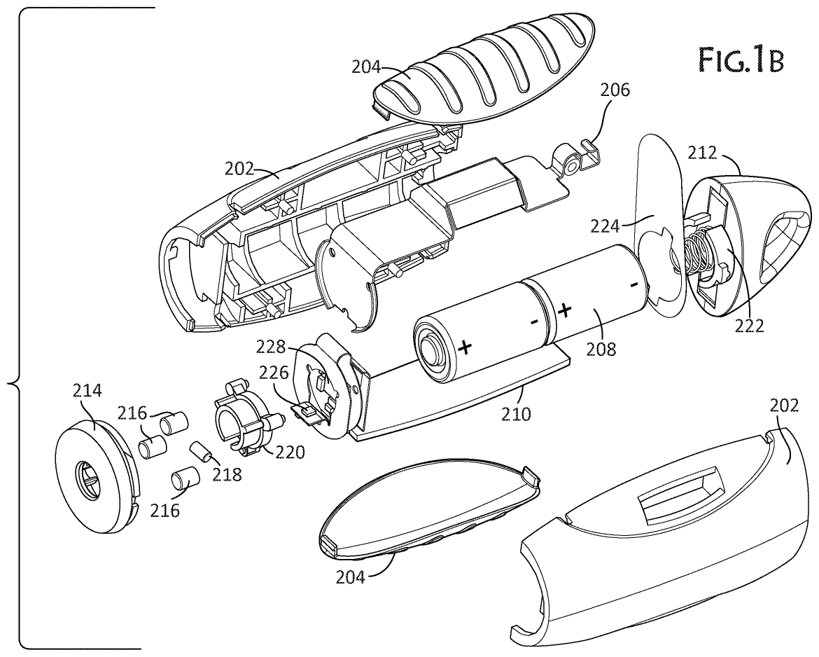

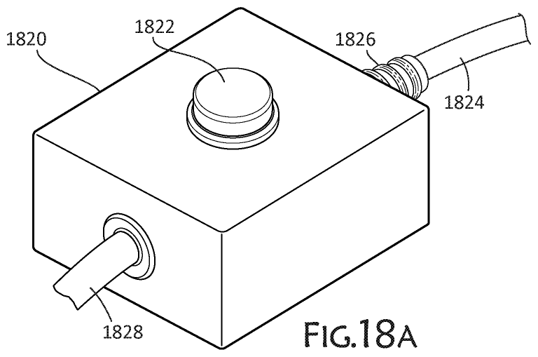

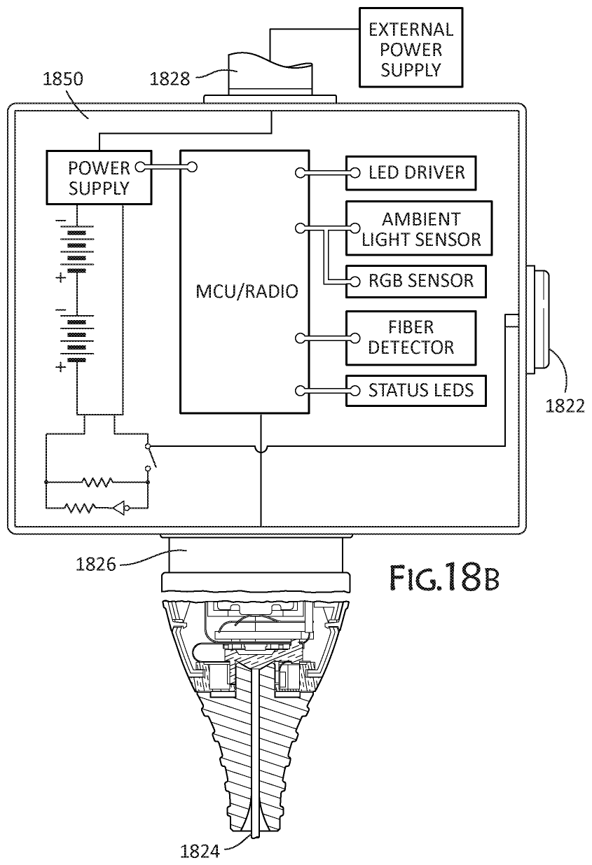

In one aspect, the LED is configured to the electronic illuminator and is controlled through a microcontroller (‘MCU’) on a printed circuit board (‘PCB’). In the example of FIG. 1A, the PCB is a Rigid-Flex PCB (‘RF-PCB’) (herein also referred to generally as a ‘PCB’) wherein part of the board is rigid and the other part is defined within a flexible ribbon, thus allowing for applications such as within the electronic illuminator housing as disclosed herein. In other examples, such as with FIG. 18B, the PCB is a standard rigid PCB configured with the MCU and sensor assemblies.

The electronic illuminator is configured to illuminate a fiber optic cable having a proximal end with a cap and a terminal end that terminates in a reflective cap. The fiber optic may be side glow or fuzzy fiber optic cable that allows light to emanate or leak to the outside, causing a glow or luminescence. The fiber optic cable is configured to run alongside a medical infusion tube, to allow a specific medical infusion tube to illuminate for that a practitioner may identify the correct port and medicine to deliver.

The electronic illuminator, in one aspect, comprises a housing, along with a rigid-flex PCB or RF-PCB or PCB, and a power source. The housing may be comprised of a polymeric material and have various metal or other heat transferring locations, effectively forming external heat sinks within the housing that connects to an internal heat sink. Further, in other aspects, the housing may be comprised of metal or a blend of polymeric material and a metal, thus forming a protective enclosure for the various assemblies and subsystems. In one aspect the housing allows the electronic illuminator to be water tight or dust proof, and in other aspects it may be rated for waterproofing for a certain period of time at specific atmospheric pressure. Rubber gaskets may align the surfaces of the housing, as well as rubber material for grip, such as textured rubber where a user may come into contact with the electronic illuminators housing. The rubber gaskets assist in water proofing, vibration, dust proofing, and may further attribute to ingress protection, allowing some examples to achieve ratings such as IP65, IP66, and IP67.

Returning to the RF-PCB, in one example it may be configured with an ambient light sensor that is operatively configured within the housing of the electronic illuminator. The ambient light sensor may be any number of makes or models, for example, it may be a sensor manufactured by Lite-On™, such as the LTR-329ALS-01. In one aspect, the flexible region of the RF-PCB allows for adjusting and aligning the ambient light sensor to offset from the LED, therefore allowing for detection of whether or not the LED is powered, along with the LED's relative intensity, and detection of environmental lux. These features incorporated with the onboard microcontroller allow for automatic light intensity configuration through the power drivers on the RF-PCB. In another aspect the ambient light sensor converts light intensity to a digital signal, such as lux, thought an analog to digital converter on the sensor, and transmits the lux value to a microcontroller. In another aspect the conversion is processed on a microcontroller on the RF-PCB, and further used to determine behavior of an electronic illuminator. Even further embodiments, the processing may occur on a microprocessor, wherein the microprocessor may be standalone, or it may be incorporated onto the microcontroller unit.

In one aspect, a sensory system for an electronic illuminator to detect the presence and color of a fiber optic cable cap or funnel cap is disclosed. A sensory system comprises various components, assemblies, and configurations disclosed herein. In one example a sensory system is configured to a light source, such as a light emitting diode (‘LED’). An LED is typically comprised of a silicon lens, a ceramic substrate, a thermal pad, a bond layer, an LED chip or microprocessor, a phosphorous layer, and a cathode. The entire construction is typically within a few millimeter package. Typically, several LED chips are packaged together to provide enough luminous flux to serve the purpose of illuminating.

In another aspect, an electronic illuminator is configured with a fiber detection assembly. Wherein the fiber detection assembly is a hall effect sensor or hall sensor. The assembly may be comprised of a plurality of magnets and a steel bar to create a tuned magnetic field, so as to resemble a key or signature that allows detection of fiber insertion into the electronic illuminator. The key or signature may be unique to a fiber line, to a color, or may be coded for other intelligence. The fiber detection assembly further having a three dimensional magnetic flux density based on at least one or more magnets configured to a fiber funnel cap.

In additional aspects, a cap color detection assembly is incorporated onto the RF-PCB of an electronic illuminator. Wherein the cap color detection assembly detects the color of the cap, or a funnel cap, or other design enabled to configure into or on an electronic illuminator to direct light along the fiber optic cable. The cap color detection assembly, in one aspect, may be comprised of an R/G/B sensor, a light emitting component and an integrated circuit or microprocessor. In further aspects, the cap color detection assembly may be positioned towards the light emitting diode, so as to receive the light radiation directly. Additionally, a cap color detection assembly may comprise a color code band detection, wherein the cap comprises a multiple band color code and the cap color detection assembly is capable of scanning the multiple band code and returning instructions, such as the color the band is coded for or additionally, whether the code also instructs things such as sound, illuminance patterns, or other system alerts.

In additional aspects, an ambient light sensor, a fiber detection assembly, and a cap color detection assembly are operatively configured to the RF-PCB, also referred to generally as a PCB, in the electronic illuminator. The three subsystems work in coordination, and may rely on one another, for example, the ambient light sensor may work in coordination with the fiber detection assembly to determine if a fiber optic cable is present, or if it has illumination running through it. Such procedures may be used to detect a failure in the fitting of the fiber optic line, or may also detect unauthentic, or not genuine configurations.

Referring now to additional aspects of the electronic illuminator. In one aspect the housing may further be comprised of a heat sink. The heat sink may be metal based or based from other heat transferable materials that allow the dissipation of heat energy from the LED, the power drivers, the microcontroller, and the various microprocessors onboard an example electronic illuminator system. Furthermore, the heat sink may be aligned with the power source, such as a battery. The battery may comprise any number of chemistries that are available to provide durational power support for the electronic illuminator.

Referring to methods herein, in one aspect a method for detecting the presence of a fiber optic line or cable in an electronic illuminator is disclosed. In one aspect, the method comprises provisioning an electronic illuminator with an ambient light sensor and a fiber detection assembly. Wherein the ambient light sensor is configured to receive light from outside of the electronic illuminator, thus in one configuration on the back side of the RF-PCB, allows for diffused environmental light to be acquired through a translucent ring or window on the exterior housing of the electronic illuminator. Next, the ambient light sensor or microcontroller unit acquires the light intensity and coverts it to a digital signal (lux). Next, the MCU determines the lux based on the digital signal. Then the hall effect sensor on the fiber detection assembly acquires a magnetic flux density. The MCU then determines a tesla value based on the magnetic flux density. Through the hall effect sensor and the ambient light sensor, the presence of a fiber optic cable may be detected with a digital signal and tesla value, or may be detected independently by either assembly or sensor. Further, when acquiring a magnetic flux density through the hall effect sensor on the fiber detection assembly, the strength of the magnetic flux may be represented based on output voltage. Lastly, the example aspect discloses alerting, by the electronic illuminator, that a fiber optic cable is present. Further, the electronic illuminator may use the subsystem for authenticating that the fiber optic cable is authentic for use with the electronic illuminator based on tesla value alone or in combination with cap color detection assembly.

In additional aspects, the electronic illuminator module may comprise an optical power sensor assembly for detecting the presence, length, and authenticity of a side scattering fiber optic cable. The optical power sensor assembly, in one aspect, utilizes time-of-flight (‘ToF’) measurement principles to detect light travel time from the proximal end to the distal end of the side scattering fiber optic cable.

In one embodiment, the optical power sensor assembly comprises a pulsed light source, such as a laser diode or high-speed LED, capable of emitting light pulses with durations in the nanosecond range. The assembly further comprises a high-speed photodetector positioned to receive reflected light from the polished interior surface of the protective end cap at the distal end of the fiber optic cable. The MCU, in coordination with timing circuitry such as a time-to-digital converter (‘TDC’), measures the elapsed time between pulse emission and detection of the reflected pulse.

In one aspect, the optical power sensor assembly utilizes phase-shift measurement techniques, wherein a modulated light signal is transmitted through the fiber optic cable and the phase difference between the transmitted and reflected signals is analyzed to determine travel time. The phase shift correlates to the optical path length of the fiber optic cable.

The detected travel time may be used by the MCU for multiple purposes, including: (1) determining fiber optic cable length to adjust LED intensity accordingly; (2) authenticating genuine fiber optic cables by comparing measured travel time against expected values stored in memory or downloaded from a repository; and (3) detecting fiber damage or disconnection when travel time falls outside acceptable thresholds.

In one example, for a side scattering fiber optic cable of approximately one meter in length, with a refractive index of approximately 1.5, the expected round-trip travel time is approximately 10 nanoseconds. The optical power sensor assembly may be calibrated with tolerance thresholds of plus or minus 2 nanoseconds to accommodate manufacturing variations.

The embodiments herein may also be incorporated into a housing of a medical infusion pump. For example, the various components, apparatus, and system may be incorporated into a medical infusion pump, whereas the housing becomes the medical infusion pump housing. Further, the heat sinks may be incorporated, or with the additional space afforded by the medical infusion pump, they may be removed all together. The following reference to figures are embodiments, any of which may include a medical infusion pump, wherein the microcontroller unit or MCU is a processor onboard the medical infusion pump.

In some aspects systems and methods for power management are disclosed. In one aspect power management within the electronic illuminator is handled by the MCU, wherein the optical detection assembly, the cap color detection assembly, and the fiber detection assembly work to provide power savings. In one aspect, the ambient light sensor

### III. With Reference to Figures

Referring now to FIG. 1A, a perspective view of an illustration of an example electronic illuminator, displaying internal components and subsystems. In the example, an electronic illuminator 100 is disclosed with a fiber funnel cap that may also be referred to as a fiber cap or funnel cap. The fiber funnel cap, in the example, may be of a specific color, wherein when inserted into the receiving unit of the electronic illuminator 100, the color is detected and the electronic illuminator 100 is set to illuminate the LED driver corresponding to cap color. This feature reduces cognitive load as it is intuitive, wherein a red cap will configure the electronic illuminator 100, through an MCU, to display a red light down the fiber optic line 108. In this example, the fiber optic line 108 being a side emitting or side scattering fiber optic line, or one with poor transmission that allows light filter outside of the directionality of the line.

Continuing, in the example, a RF-PCB 104 is disclosed, wherein the flex portion is folded unto itself, forming a location for the R/G/B sensor of a cap color detection system. The folds allow for blocking of the ambient light sensor from the onboard LED of the electronic illuminator, wherein the ambient light sensor acquires environmental lux from the clear housing, also referred to as a translucent ring 102, at the proximal end of the electronic illuminator 100. In other aspects the translucent ring 102 may be opaque or may have a window in it that allows for environmental light. In further embodiments the light from the side emitting fiber optic line may be used to determine environmental lux. Additionally, the magnets supplied for the hall effect sensor forming the fiber detection assembly are embedded within the translucent ring 102, or positioned near the translucent ring 102, to form a magnetic field.

A battery 106 is disclosed along with an internal heat sink 112, wherein the battery powers the electronic illuminator's various assemblies and the internal heat sink 112 works to dissipate heat to the external heat sink on the housing. An LED assembly 110 is positioned to connect with the fiber funnel cap to project light through the side emitting fiber optic line 108. An end cap 114 to the electronic illuminator holds the batteries in place and may further house a communications module or assembly as well as an antenna. With regard to the battery 106, the electronic illuminator, in one aspect, may work in coordination with the ambient light sensor and the cap color detection assembly or the fiber detection assembly to regulate usage of power and to form a swarm of sensors for intelligent power management. In one aspect, the ambient light sensor detects lux in the environment and controls LED power output from the power driver to conserve energy. Further, the cap color detection assembly may detect the fiber funnel cap is not engaged and thus automatically turn the system off. Similarly, the fiber detection assembly may detect an absence of a fiber and a fiber funnel cap, therefore turning the power off until the fiber funnel cap is attached.

Referring now to FIG. 1B, an exploded view of an illustration of an example electronic illuminator. In the example a housing 202 has locations for an external heat sink 204, wherein the external heat sink 204 is in thermal connection with an internal heat sink 206. The internal heat sink 206 is designed to contact equipment such as the LED power drivers, the LED, and other integrated circuits or microcontrollers, including processing units, so as to reduce heat build-up and control thermals within the tight enclosure. The housing of the example electronic illuminator is further configured with an end cap 212, the end cap having a negative terminal 222. The end cap 212 secures the batteries in place and allows for rapid exchange of batteries. In the example, the end cap 212 has split paper 224 to separate the contact of the batteries and allow for an extended shelf life of the electronic illuminator. Additionally, a translucent ring 214 forms the proximal end or end nearest the fiber optic line, wherein the translucent ring 214 allows light to reach an ambient light sensor. Similarly, in additional embodiments a window to the environment may be provided, wherein the translucent ring is opaque or not translucent and a window within the ring may allow for observing the lux within the environment.

Various examples disclosed herein contain reference to the electronic illuminator, and are identified in FIG. 1B. In one aspect the illuminator is housed within a front shell and a back shell, also referred to as a housing 202. The housing is often made of a polymer but can be made of other materials such as a metal casing. The housing of the electronic illuminator serves to protect the assemblies, sensors, and controllers, as well as provide positioning of said components, and account for size, durability, and ease of transmission of RF signals. An electronic illuminator end cap 212 secures a lithium ion or other battery in place within the shell or housing 202 of the electronic illuminator. The end cap 212 is equipped to receive a piece of split paper 224 to break the current and allow for longer shelf life and storage of the electronic illuminator. In additional aspects an internal heat sink 206, which is integrated along the PCB 210 and/or microcontroller and battery supply or batteries, the internal heat sink 206 is then connected through a high thermal conductive material to the metal side covers, or external heat sinks 204 to further dissipate heat. In other aspects the metal side cover is fully formed to the internals of the electronic illuminator and provides a passive environmental cooling complex.

In another aspect of the example of FIG. 1B, power is supplied from a plurality of batteries 208, which may be of lithium chemistry, or other chemistry, to allow for powering an electronic illuminator. Additionally, the electronic illuminator's batteries 208 may be charged wirelessly or through a uniform serial bus connection such as a USB-A, USB-B, USB-C, or any micro variants thereof. The RF-PCB 210, also known herein as a PCB, contains a microcontroller, along with the various assemblies and sensors. The RF-PCB 210 forms a folded structure to allow for unique positioning of sensors and assemblies so as to allow for optimal operation. In one aspect, the ambient light sensor is formed to the backside of the RF-PCB 210, so that ambient light from the environment, penetrating through the translucent ring 214 is the only perceived light. This allows for automatic adjustment by the MCU for controlling light intensity. For example, if the surrounding environment is dark, the amount of lux produced by the LED can be lower, as the overall system needs do not require a high lux operation, thus conserving battery life and equipment from excess heat and usage.

The LED assembly 228 on the RF-PCB 210 is configured with a lens 220, wherein the lens 220 is situated to receive the fiber for illumination. The fiber side, in one aspect, is equipped with a fiber funnel cap, that configures to the translucent ring 214 and is held in place by a locking mechanism or through magnetic force and use of magnets, such as a magnet assembly 216, or other configurations of magnets. The R/G/B sensor 226, comprising the cap color detection assembly, is disclosed facing inwards toward the receiving orifice of the fiber funnel cap. Additionally, the magnet assembly 216, in coordination with the steel bar 218 provides a magnetic flux key or signature that may be utilized for fiber detection, as well as authentication and security. Wherein the electronic illuminator may be configured to authorize use of a signature or flux key.

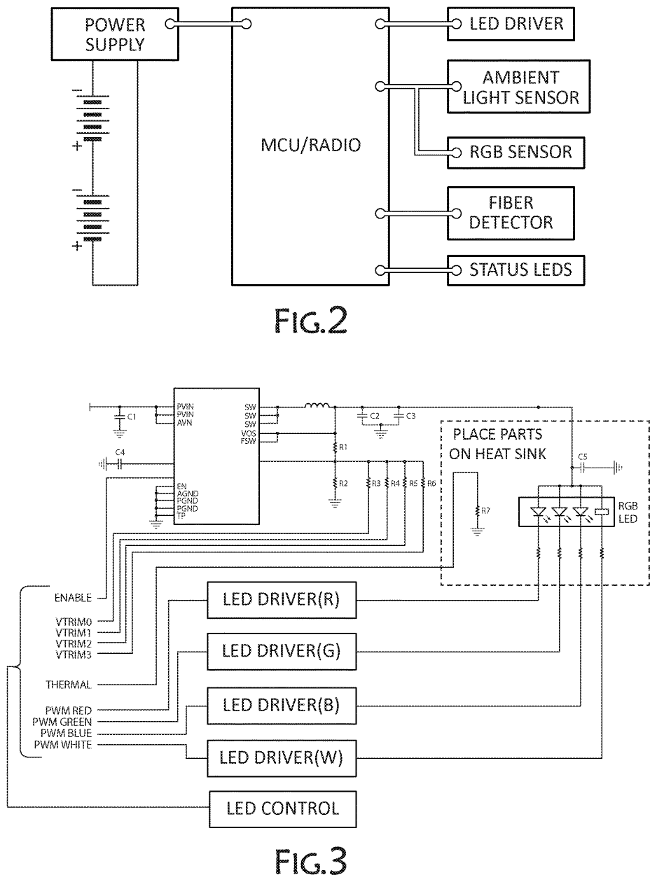

Referring now to FIG. 2, a schematic diagram of an example of an electronic illuminator's internal components and subsystems. In one aspect a microcontroller unit or MCU is configured to a power supply such as a battery or may be directly powered through a USB connection to a power source external to the device. Similarly, the power supply may be adapted to receiving power wirelessly through such standards as Qi charging. The schematic of FIG. 2 is an overall generalization of an example of an electronic illuminator, including components such as an LED driver, ambient light sensor R/G/B sensor (which forms a cap color detection assembly), a fiber detection assembly (based in part on a hall sensor), status LED's, and a power supply.

The various components, assemblies, sensors, and subsystems may be in communication utilizing an inter-integrated circuit (‘I²C’) interface for intra-board communication. Additional communications protocols such as wireless, Bluetooth™, and other radio standards may be additional chipsets configured with the onboard MCU.

Turning now to FIG. 3, a schematic diagram of an example of an electronic illuminator's LED power system drivers. In one aspect the power drivers illuminate LED's for varying color frequency. In the disclosed example, a red, green, blue, and white driver powers the various aspects of color. Due to heat or radiance or thermals from the drivers powering the LED components, a heat sink may be applied, such as the one disclosed in FIG. 1B, wherein the heat is diffused along an electronic illuminator, and dispersed exterior thereof through a heat sink mounted externally. Thus, the present embodiment is an internal metallic heat sink in thermal communication with an external facing metallic heat sink. Additional configurations of the LED drivers, as well as LED powering assembly and the chipset are disclosed herein.

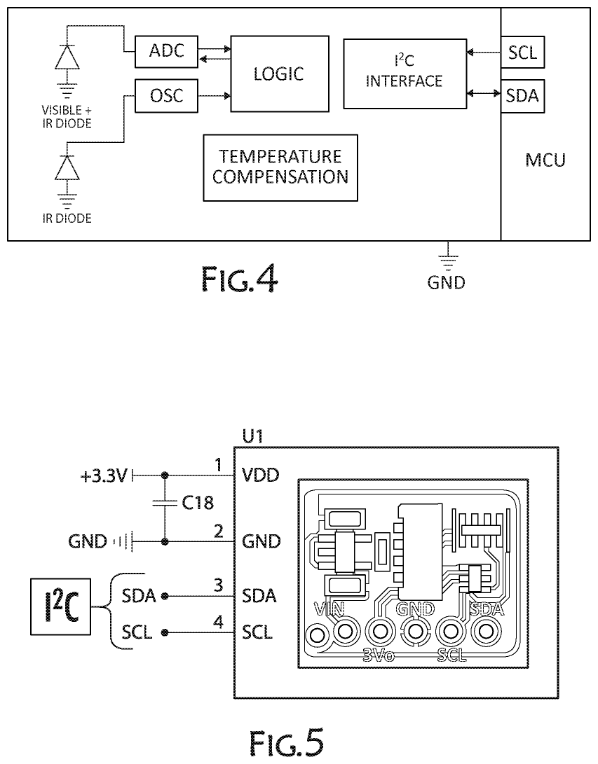

Referring now to FIG. 4, a block diagram of an example of an electronic illuminator's ambient light sensor. In one example the ambient light sensor converts light intensity to a digital output signal capable of direct inter-integrated circuit—I²C. With reference to FIG. 4, the Analog to Digital Converter (‘ADC’), is configured to the visible IR diode and IR diode, wherein the logic is responsible for converting. Continuing, in the example, the I²C interface is displayed in connection with the Serial Clock (‘SLC’) and serial data. Thereby, I²C being a synchronous, multi-controller, multi-target, packet switched, single-ended serial communications bus. I²C uses only two bidirectional open collector or open drain lines, serial data line and serial clock line, pulled up with resistors. Typical voltages used are +5 V or +3.3 V, although other voltages are common.

In the example of FIG. 4, within operating temperatures within a range of 30° Celsius to 700 Celsius, the example ambient light sensor may perceive 6 dynamic ranges from 0.01 lux to 64,000 lux, and automatically reject 50/60 Hz lightings flicker. Thus, in the example surface mount package, the ambient sensor converts light intensity to a digital output signal that is capable of direct I²C interface with an MCU.

Turning now to FIG. 5, a schematic diagram of an example of an electronic illuminator's ambient light sensor chipset. There are three main types of ambient light sensors, namely, photodiodes, photonic ICs, and phototransistors. In principle they work along the same lines of converting light to voltage or current, and using the voltage or current for modes of operation. Typically, light enters the photodiode, wherein a thin layer allows photons to pass through it into a depletion region where a pair of electron holes are formed. The electric field across the depletion region causes electrons to be swept into an N layer. In some aspects, the ambient light sensor provides linear response over a wide dynamic range from 0.01 lux to 64,000 lux. The lux (symbol: lx) is the SI derived unit of illuminance, measuring luminous flux per unit area. It is equal to one lumen per square meter. In photometry, this is used as a measure of the intensity, as perceived by the human eye, of light that hits or passes through a surface.

Illuminance is analogous to the radiometric unit watt per square meter, but with the power at each wavelength weighted according to the luminosity function, a standardized model of human visual brightness perception. Illuminance is a measure of how much luminous flux is spread over a given area. One can think of luminous flux (measured in lumens) as a measure of the total “amount” of visible light present, and the illuminance as a measure of the intensity of illumination on a surface. A given amount of light will illuminate a surface more dimly if it is spread over a larger area, so illuminance is inversely proportional to area when the luminous flux is held constant.

The illuminance provided by a light source on a surface perpendicular to the direction to the source is a measure of the strength of that source as perceived from that location. Like all photometric units, the lux has a corresponding “radiometric” unit. The difference between any photometric unit and its corresponding radiometric unit is that radiometric units are based on physical power, with all wavelengths being weighted equally, while photometric units take into account the fact that the human eye's image-forming visual system is more sensitive to some wavelengths than others, and accordingly every wavelength is given a different weight. The weighting factor is known as the luminosity function.

The lux is one lumen per square meter (lm/m2), and the corresponding radiometric unit, which measures irradiance, is the watt per square meter (W/m2). There is no single conversion factor between lux and W/m2. There exists a different conversion factor for every wavelength, and it is not possible to make a conversion unless one knows the spectral composition of the light. The peak of the luminosity function is at 555 nm (green); the eye's image-forming visual system is more sensitive to light of this wavelength than any other. For monochromatic light of this wavelength, the amount of illuminance for a given amount of irradiance is maximum: 683.002 lux per 1 W/m2; the irradiance needed to make 1 lux at this wavelength is about 1.464 mW/m2. Other wavelengths of visible light produce fewer lux per watt-per-meter-squared. The luminosity function falls to zero for wavelengths outside the visible spectrum.

For a light source with mixed wavelengths, the number of lumens per watt can be calculated by means of the luminosity function. In order to appear reasonably “white”, a light source cannot consist solely of the green light to which the eye's image-forming visual photoreceptors are most sensitive, but must include a generous mixture of red and blue wavelengths, to which they are much less sensitive.

This means that white (or whitish) light sources produce far fewer lumens per watt than the theoretical maximum of 683.002 lm/W. The ratio between the actual number of lumens per watt and the theoretical maximum is expressed as a percentage known as the luminous efficiency. For example, a typical incandescent light bulb has a luminous efficiency of only about 2%. In reality, individual eyes vary slightly in their luminosity functions. However, photometric units are precisely defined and precisely measurable. They are based on an agreed-upon standard luminosity function based on measurements of the spectral characteristics of image-forming visual photoreception in many individual human eyes.

In the example of FIG. 5, an ambient light sensor, configured as an ambient light sensor subsystem is integrated into the rigid-flex printed circuit board. By integrating the ambient light sensor, the electronic illuminator possesses the ability to visualize or detect the connection of the fiber feed by determining environmental lux, and the change in lux once the fiber is configured. An increase of lux within the housing, without an increase in the lux externally to the electronic illuminator may single that the onboard LED is active, but transmission through the fiber optic cable is not occurring. Similarly, with ambient lux increased, from powering on of the LED drivers, it may signal that the electronic illuminator is operating as intended.

In other aspects, there exists a translucent ring at the proximal location of the illuminator to detect the ambient light difference from that of the LED's of the electronic illuminator. In one aspect, the ambient light sensor is placed on a flexible region of a RF-PCB in a location that is shaded from luminance of the fiber source. If the ambient light sensor detects ambient light with no fiber attached it can switch the microcontroller into low power mode. The ambient light sensor, in other aspects, assists with power control and sleep wake. In other aspects, the ambient light sensor detects errors within the LED or within the electronic illuminator. The ambient light sensor, in additional embodiments is equipped to detect occlusion of the signal of the electronic illuminator and to alert or otherwise inform users of an issue with the electronic illumination system.

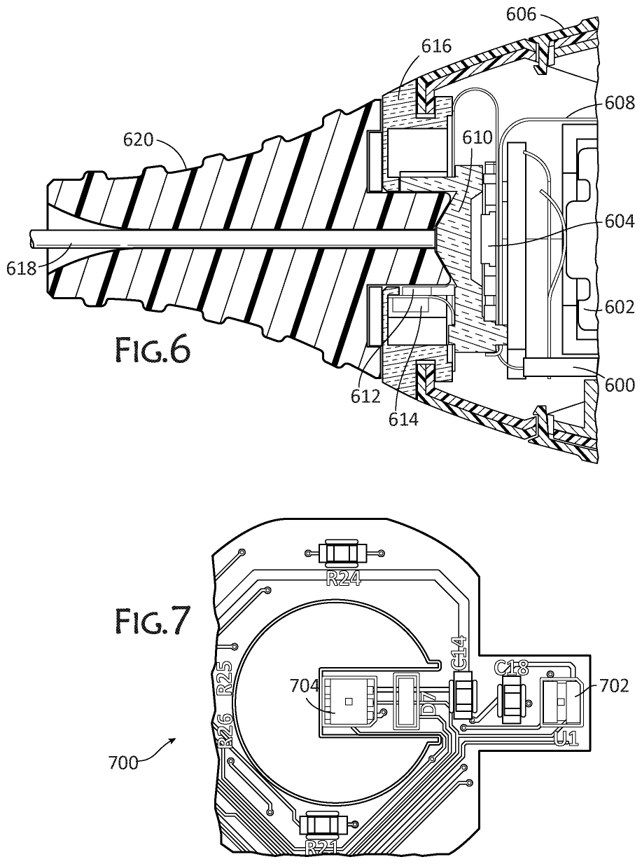

Referring now to FIG. 6, an illustration of an example of an electronic illuminator in cross section, disclosing the ambient light sensor configuration within the electronic illuminator. In the example, the ambient light sensor is configured in a region shaded from the LED 604 of the electronic illuminator. The ambient light sensor 614 is directed to receive lux or light from the environment through the translucent ring 616. In additional embodiments, the translucent ring 616 may have a filter to shade certain lux ranges so as to enable optimal performance. In other embodiments the translucent ring 616 may be non-translucent and have a window to the exterior for accumulating environmental lux as part of a power savings algorithm and automatic adjustment of LED power. For example, if the ambient light sensor 614 detects ambient light with no fiber attached it can switch the microcontroller or MCU into low power mode by communicating across the PCB 600. The ambient light sensor 614, in other aspects, assists with power control and sleep wake functionality, as well as instructing with other assemblies the proper usage by identifying whether or not the luminosity is enough to impact the environmental lux. When the power source is a battery 602, the algorithm may have conservative parameters versus connected directly to a power source such as a wall outlet. In other aspects, the ambient light sensor detects errors within the LED or within the electronic illuminator by detecting no light is emitting through the lens 610. The ambient light sensor, in additional embodiments, is equipped to detect occlusion of the signal of the electronic illuminator and to alert or otherwise inform users of an issue with the electronic illumination system.

Continuing, in FIG. 6, the R/G/B sensor 612 forming the cap color detection assembly is facing towards the receiving orifice of the fiber 618 and fiber funnel cap 620. The R/G/B sensor being equipped to read the outer surface of the inserted portion of the fiber funnel cap 620. Further, the R/G/B sensor is able to detect the color of the fiber funnel cap 620 for instructing the MCU to power LED drivers for a specific color or for other computational routines. The assemblies mentioned herein may generate heat, the internal heat sink 608 is positioned across those components and in thermal communication with the external heat sink 606 that in this example forms part of the housing of the electronic illuminator.

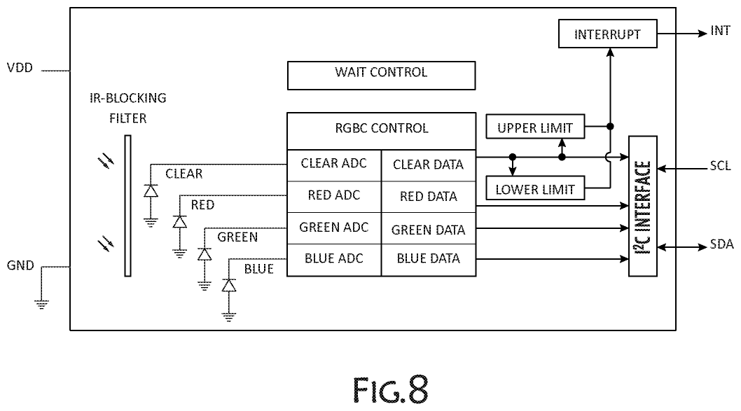

Referring now to FIG. 7, an illustration of an example of an electronic illuminator's Rigid-Flex PCB 700, wherein the flex portion is disclosed with the ambient light sensor 702 and the cap color detection assembly configured with an RIG/B sensor 704. The cap color detection assembly having the capability to read the cap color of a fiber funnel cap, to program the LED power drivers to illuminate a specific range of LED light to match the fiber funnel cap. In one aspect, the RGB sensor may be color light sensing with an IR blocking filter and with high sensitivity. Example manufacturers include Misumi™, Excelitas™, Ams™, and include low power options with high sensitivity.

Examples of Benefits and Features for an RIG/B Sensor are disclosed in the table below:

R/G/B Sensor

| Benefits | Features |
|---|---|
| Enables accurate color and | Red, Green, Blue (RGB), |
| light sensing measurements under | and Clear Light |
| varying lighting conditions by | Sensing with IR blocking |
| minimizing IR and UV spectral | filter |
| component effects | Programmable analog gain |
| | and integration time |
| | 3,800,000:1 dynamic range |
| | Very high sensitivity |
| Programmable interrupt pin | Maskable interrupt |
| enables level-style interrupts when | Programmable upper and |
| pre-set values are exceeded, thus | lower thresholds with persistence |
| reducing companion micro- | filter |
| processor overhead | |
| Enabling a low-power wait- | Power management |
| state between RGBC | Low power-2.5 μA sleep |
| measurements to reduce average | state |
| power consumption | 65 μA wait state with |
| | programmable wait state time from |
| | 2.4 ms to >7 seconds |
| Digital interfaces are less | I²C fast mode compatible |
| susceptible to noise | interface |
| | Data rates up to 400 kbit/s |
| | Input voltage levels |
| | compatible with VDD or 1.8 VBUS |

In one aspect, an R/G/B sensor, as part of the cap color detection assembly is configured to read a multiple band code, wherein the bands may be coded or preprogrammed within the MCU to illuminate at a specific spectrum, such as to produce a specific color of light. The multiple band code may be transcribed as rings on a fiber funnel cap or along the fiber line, that when engaged with the electronic illuminator allows reading of the multiple band code and transmission from the cap color detection assembly to an MCU for signaling or communicating to the LED power drivers. In other aspects, a coded SKU may be read by the cap color detection assembly and similarly transmitted to the LED power drivers.

Referring now to FIG. 8, a block diagram of an example of an electronic illuminator's R/G/B sensor forming a cap color detection assembly. In the block diagram, the R/G/B control stack shows the architecture of the integrated circuit. Further, the RIG/B sensor is in interface communication with an MCU on a RF-PCB. The configuration is one of many, other examples may include additional components or configurations. For example, a multiple band code reader may contain additional elements.

In one aspect, a cap color assembly utilizes an RIG/B sensor to identify the cap color, wherein once acquired, communicates with an MCU which in turn instructed LED power drivers to illuminate for the specific color. In other aspects, a cap color detection assembly may be coded to specific instructions, such as to illuminate with a pattern, or to indicate expected luminosity outside of the ambient light sensor, to play an audio signal, or other cognitive aspect such as identifying with medical fluid treatment. In one aspect, a red cap or red band pattern may be programmed for blood products, or blood infusion. Whereas a green cap or green band pattern may be programmed for nutrients, and blue for saline, these are but a few possibilities with the systems and methods disclosed herein.

Referring now to FIG. 9, a schematic diagram of an example of an electronic illuminator's RIG/B sensor forming a part of the cap color detection assembly. In the example of FIG. 9, a schematic for one embodiment of an R/G/B sensor is disclosed. The MCU on the RF-PCB in communication through the I²C interface. In one aspect the cap color detection sensor minimizes IR and UV spectral component to produce accurate color measurements. In another aspect the cap color detection detects the cap color of the fiber funnel cap on the side scattering fiber optic line. In other aspects the cap color detection assembly detects the color of the fiber funnel cap and registers the color with the microcontroller or MCU. In this example, the microcontroller determines what color the fiber funnel cap consists of and signals to the LED driver to illuminate the side scattering fiber optic line with the specific color. The cap color detection assembly is enabled to scan for accurate color under varying conditions, ranging from ICU room lighting to patient care and resting state lighting. Furthermore, the fiber funnel cap color may serve as a watermark, proprietary colors may be selected, and or techniques of encoding the fiber funnel caps with color properties for counterfeiting prevention and authentication. Furthermore, the cap color detection assembly is capable of SKU identification, identifying aspects of readable codes such as bar codes, QR codes, band codes, color codes or other patterns of identifying information. Such identification allows for authenticating and verifying medical equipment, which in turn helps reduce risk of patient harm, and allows the system to operate in normal fashion.

In another aspect, the cap color detection assembly comprises a photoelectric sensor arrangement instead of or in addition to the R/G/B sensor. The photoelectric sensor arrangement may comprise a plurality of photodiodes, each filtered to respond preferentially to a different wavelength band corresponding to primary colors. For example, three photodiodes with red, green, and blue optical filters may be arranged adjacent to the receiving orifice of the electronic illuminator.

In one embodiment, the photoelectric sensor comprises a broadband photodiode paired with a white LED illumination source. The cap color detection assembly cycles through illumination with red, green, and blue LED components sequentially, and the photodiode measures reflected light intensity during each illumination phase. The MCU compares the relative intensities to determine the cap color.

In another embodiment, the cap color detection assembly utilizes a color-sensitive photodiode array, wherein multiple photodiodes with different spectral sensitivities are integrated into a single package. The ratio of outputs from the different photodiodes indicates the dominant wavelength of reflected light from the fiber funnel cap.

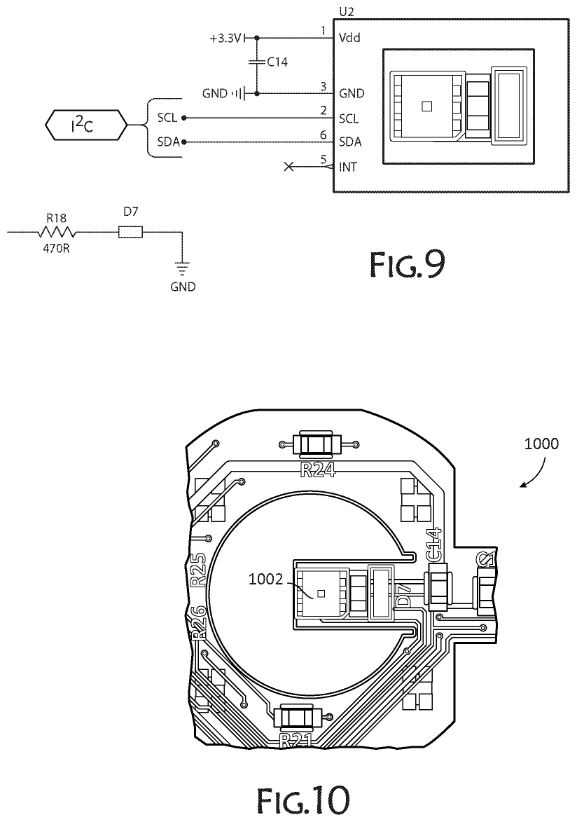

Referring now to FIG. 10, an illustration of an example of an electronic illuminator's Rigid-Flex PCB, wherein the flex portion is disclosed with the cap color detection assembly 1000. In one aspect the R/G/B sensor 1002 has an IR filter and white LED module. An example of an R/G/B sensor is the TCS34725, manufactured by TAOS™ (Texas Advanced Optoelectronic Solutions). In one aspect the R/G/B sensor 1002 returns and processes analog to digital values for a sensed object, such as a cap or band pattern. Further, in some aspects, the R/G/B sensor 1002 of the cap color detection assembly 1000 also allows for ambient light sensing for functions such as power savings, in use, security, and authentication. In one aspect the R/G/B sensor 1002 contains an 3×4 photodiode array and four analog to digital converters that integrate the photodiode, data registers, a state machine, and an I²C interface.

In additional aspects a watermark, or color array, for a series of color bands may be used for security and authentication. In one aspect a series of banded color codes is placed and read by the cap color detection assembly within the electronic illuminator. If the color code is a match the electronic illuminator functions, if the color code is not a match the electronic illuminator provides notification. Notification can consist of a signal or transmission, or other notification that the fiber is either not genuine, or is inserted incorrectly, or there is a failure within the system. Additionally, in another aspect, the color bands or watermark may also provide input to the electronic illuminator regarding the LED transmission color to illuminate the fiber line with.

In one aspect, the cap color detection assembly is configured to read a multiple band code disposed on the fiber funnel cap. The multiple band code comprises a series of circumferential color bands arranged along the axial length of the fiber funnel cap's insertion portion. Each band has a defined width, for example between 1 mm and 3 mm, and is separated from adjacent bands by a defined gap, for example between 0.5 mm and 1 mm.

In one embodiment, the multiple band code comprises three to eight color bands, wherein each band is selected from a palette of distinguishable colors including red, green, blue, yellow, cyan, magenta, white, and black. The specific sequence of colors encodes information such as: (1) the intended illumination color for the LED driver; (2) a product identification code; (3) a lot number or manufacturing date; and (4) authentication or anti-counterfeiting data.

The R/G/B sensor of the cap color detection assembly reads the multiple band code by scanning sequentially as the fiber funnel cap is inserted into the electronic illuminator. In one aspect, the MCU samples the R/G/B sensor output at a defined rate, for example 100 Hz to 500 Hz, as the cap traverses past the sensor position. The MCU's firmware processes the sampled color values to identify transitions between bands and decode the color sequence.

In another embodiment, the fiber funnel cap is rotated during insertion, and the R/G/B sensor captures color data as bands pass the sensor's field of view. The rotational movement may be achieved by helical grooves in the electronic illuminator's receiving orifice that impart rotation to the cap during insertion.

In one example, a three-band code using red, green, and blue colors provides 27 unique combinations, sufficient to encode illumination color plus additional control data. A five-band code provides 243 unique combinations, enabling product identification and authentication features.

The MCU firmware includes a lookup table that maps decoded band sequences to corresponding actions, such as activating a specific LED driver, selecting a particular color to illuminate, setting an illumination pattern, or rejecting an unrecognized or counterfeit fiber funnel cap with an alert signal.

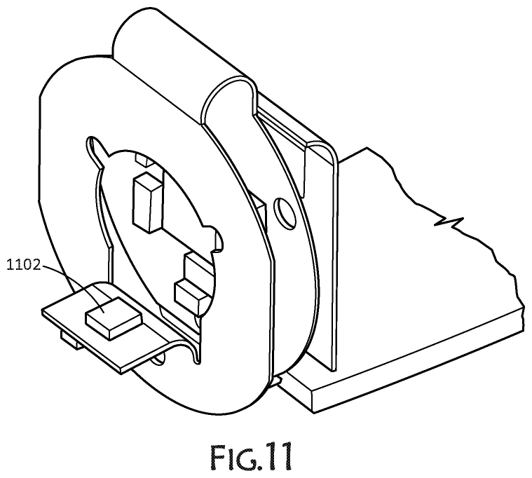

Referring now to FIG. 11, an illustration of an example of an electronic illuminator, disclosing the cap color detection assembly configuration within the electronic illuminator. In the example view the fiber funnel cap is disclosed wherein it is configured with the electronic illuminator. The R/G/B sensor 1102 of the cap color detection assembly is configured to interface with the cap of the fiber optic line. Further, the position of the R/G/B sensor 1102 also accounts for reading of a unique band code or code on the cap side or funnel cap that codes for a specific light, or for authentication, or additional features as programmed within the system. In one aspect, the multiple band code identifies red and the power driver of a red LED is turned on.

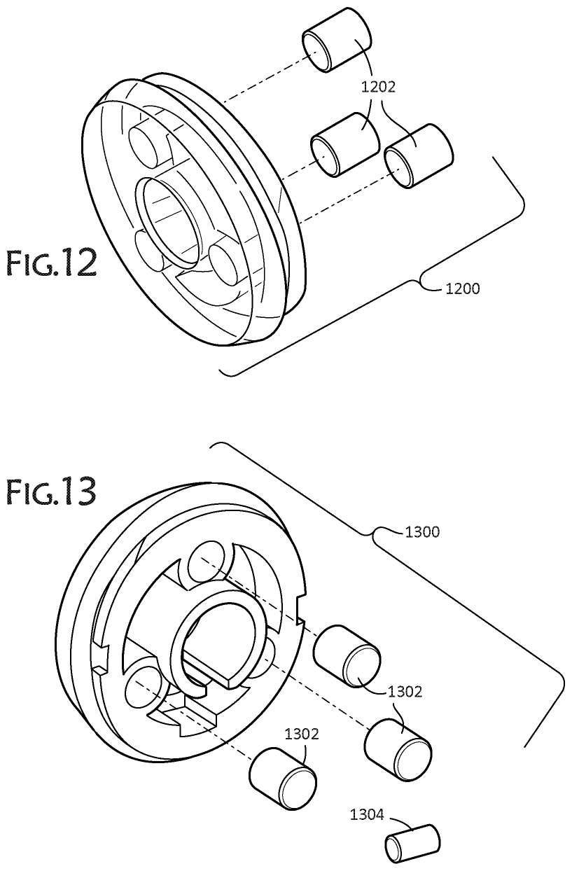

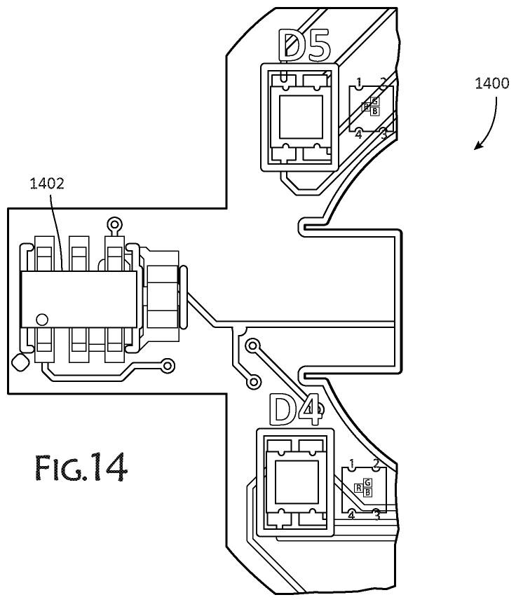

Referring now to FIG. 12, an illustration of an example translucent cap 1200 and magnet array 1202 for an electronic illuminator, the configuration provides aspects of the fiber detection assembly. The cap may be translucent and work in coordination with the optical sensor disclosed above, for sensing environmental light and light leakage from the side scattering fiber optic line. The translucent cap 1200 is fitted to the electronic illuminator forming a part of the housing. Disclosed within FIG. 12 is positioning for a plurality of magnets to form a magnetic field. Such field may be utilized for authentication, activation, security, and transmission of information utilizing a specific flux key or signature.

Similarly, referring to FIG. 13, an additional illustration of an example translucent cap and magnet array 1302 with a steel bar 1304 for an electronic illuminator, the configuration provides aspects of the fiber detection assembly. The steel bar 1304 provides tuning of magnetic field to allow for tuning of specific fields, allowing for more than authentication, but having different flux keys or signatures result in different effects, such as programmed to a specific color, or pattern for the LED driver. With regard to authentication, the steel bar 1304 may also be a magnetic bar and it is utilized in tuning, the electronic illuminator may also not require a steel bar 1304 and the system may operate with a three dimensional magnetic flux based on the magnet array 1302, wherein the key or signature is determined by the metallic plate on the funnel cap being engaged with the electronic illuminator.

In one specific embodiment, the fiber detection assembly comprises three cylindrical neodymium magnets arranged in a triangular pattern around the receiving orifice of the electronic illuminator. Each magnet has a diameter of approximately 3 mm and a height of approximately 2 mm, with a magnetic strength of approximately N35 to N42 grade. The magnets are oriented with alternating polarities to create a distinctive three-dimensional magnetic flux pattern.

The steel bar, comprising low-carbon steel or mu-metal with high magnetic permeability, is positioned across the diameter of the receiving orifice, approximately 2-3 mm from the magnet plane. The steel bar has a length of approximately 10 mm and a cross-section of approximately 1 mm by 1 mm. The steel bar shapes and concentrates the magnetic flux to create a defined signature detectable by the Hall sensor when a corresponding metal plate on the fiber funnel cap enters the magnetic field.

Referring now to FIG. 14, an illustration of an example fiber detection assembly, wherein the Rigid-Flex PCB 1400 is disclosed with a Hall sensor 1402. A Hall sensor also known as a Hall-effect sensor is a device that measures the magnitude of a magnetic field. A Hall sensor's output voltage is directly proportional to the magnetic field strength through it. Hall sensors are used for proximity sensing, positioning, speed detection, and current sensing applications. Frequently, a Hall sensor is combined with threshold detection, so that it acts as and is called a switch. Commonly seen in industrial applications, Hall sensors are also used in consumer equipment and medical applications; for example, some computer printers use Hall sensors to detect missing paper and open covers. They can also be used in computer keyboards, an application that requires ultra-high reliability. An example of a Hall sensor is the US1881 by Melexis™ that is based on mixed signal CMOS technology. In one aspect, the Hall sensor is equipped with high magnetic sensitivity, has an operating voltage of 3.5V up to 24V, and a low current consumption.

In one aspect, the Hall sensor 1402 on the electronic illuminator detects the presence of the fiber line through a series of magnets, such as a magnetic assembly, that is placed on the front cap or translucent ring of the electronic illuminator. In one embodiment the electronic illuminator creates a 3D magnetic flux density that is capable of sensing to +/−160 mT. In other embodiments a range exists over +/−160 mT. The Hall sensor is equipped with a programmable flux resolution to 65 uT. Therefore, enabling position detection and X-Y angular and fiber orientation and measurements. The three dimensional magnetic flux is also known as a magnetic flux key or signature, and such signature can be used for authentication and verification of the illuminating infusion line or the fiber line. Further, in other aspects, the Hall sensor 1402 controls the power supply and sleep wake functionality. The hall sensor 1402 is further equipped to provide energy saving aspects by controlling functionality of on/off, sleep/wake, rest state of a microcontroller. In other aspects, the Hall sensor 1402 enables device security through detection of a flux key or signature.

In the example of FIG. 14, in one aspect of the electronic illuminator a plurality of magnets for a magnetic field that can be registered by onboard sensors, such as a hall sensor, the flux may be used to verify the device is in use or other aspects such as security and authentication. In such a scenario the magnetic field is tuned to a flux key or signature so as to authentic the device. In one aspect a steel pin is utilized to adjust the flux field, in another a different material capable of disturbing magnetic force is used. In one aspect the steel pin is set to match the signature of a specific funnel color of illuminating fiber optic line. In another aspect the steel pin or other magnetic flux disturbing device is positioned for specific voltage readings from the Hall sensor. In such an embodiment authenticating the various attachment fiber optic lines can be observed. Further, in additional embodiments the sleep wake function of the microcontroller may be activated by the Hall sensor, thus allowing power conservation.

Continuing with FIG. 14, in one aspect the fiber funnel cap may be a specific color that designates the color of LED light the LED power driver will illuminate within the electronic illuminator. The fiber funnel cap is located proximal to the electronic illuminator, and at the opposite end of where the fiber line terminates. The distal end of the fiber line may have a protective distal cap. In one aspect, the fiber funnel cap may be equipped with a metal plate to match and verify a specific signature (magnetic flux key, flux signature) wherein the electronic illuminator's Hall sensor, as part of the fiber line detection assembly, can sense and acknowledge the signature or voltage change. In some aspects, the fiber side cap, equipped with a magnetic plate is used for authentication and verification. In other aspects, the fiber side cap is utilized for providing instruction to the electronic illuminator. In additional aspects, an antenna may be placed on the proximal cap cover of the fiber line, in which the microcontroller may send a radio pulse and receive a signal, this embodiment may be tied to an optical power sensor, or the microcontroller, or both.

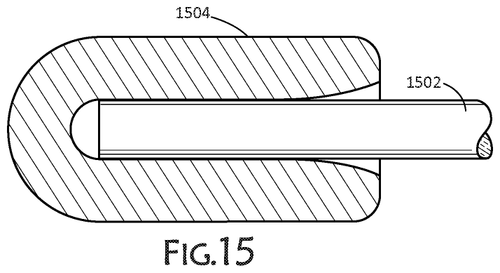

Referring now to FIG. 15, an illustration a distal end cap of a side scattering or side emitting fiber optic line. In the example the protective end cap 1504 for the side scattering fiber optic line 1502 has a polished or painted surface that is highly reflective of light. The polished or painted surface allows for reflection, similar to a mirror, wherein the programmed MCU on the PCB may initiate a sequence of flashes or pulses for authentication or verification. These pulses or flashes emanate from the LED on the electronic illuminator, travel the length of the side scattering fiber optic cable, and are reflect back from the protective end cap into the electronic illuminator where the ambient light sensor detects the pulses or flashes and interprets the signal. Additionally, a routine of pulsed light may be used for signaling the length of the fiber optic line 1502, for regulating intensity of the LED and thus controlling power drivers and saving power. If the pulsed light is not returned at a specific strength of lux it may be determined the side scattering fiber optic line is too long for usage or it may adjust power of the LED in order to broadcast along a longer fiber optic cable. This may work in coordination with the ambient light sensor, disclosed previously, to regulate power output based on the environmental light and length of the fiber optic line 1502.

Continuing, the protective end cap 1504, with a polished surface, is also made of a resilient material such as a hard plastic or metal that allows for protection of the fiber optic line 1502 so it does not fray or come into contact with patients. Additionally, the protective end cap is smooth and made to be non-abrasive and easy to clean, with no openings or otherwise which allows for prevention of bacterial growth and reusability.

In one aspect, the one-line antenna sensor comprises a passive radio-frequency identification (‘RFID’) antenna integrated into or adjacent to the protective end cap. The antenna may be a printed conductive trace on a flexible substrate wrapped around or embedded within the protective end cap. In one example, the antenna operates in the high-frequency (‘HF’) band at 13.56 MHz or the ultra-high-frequency (‘UHF’) band at 860-960 MHz.

The RFID antenna is coupled to a passive RFID chip that stores identification data such as a unique serial number, manufacturing information, or authentication codes. When interrogated by an RFID reader positioned in the electronic illuminator module or medical infusion pump, the antenna harvests energy from the interrogating RF field and powers the RFID chip to transmit stored data back to the reader.

In another embodiment, the one-line antenna sensor comprises a near-field communication (‘NFC’) tag integrated into the protective end cap. The NFC tag communicates with an NFC reader in the electronic illuminator module when the fiber optic cable is routed within proximity of the reader, for example within 4 centimeters.

In one aspect, the one-line antenna sensor enables determination of fiber optic cable length by encoding the cable's manufactured length in the RFID or NFC tag's memory. The electronic illuminator module reads this length value and adjusts LED intensity accordingly to ensure consistent illumination along the cable's length.

Further, the protective end cap 1504 may be equipped with a one line antennae that is utilized for communicating over radio frequency, thus adding an additional layer of communication to the fiber optic line. The one line antennae acts as a passive wireless antenna and may be used for determining fiber optic line length or for verification and authentication.

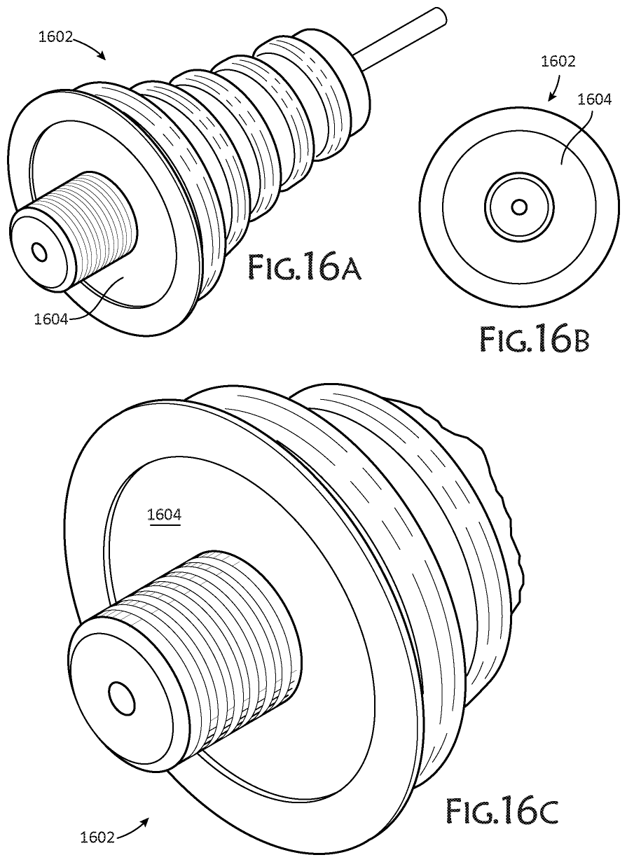

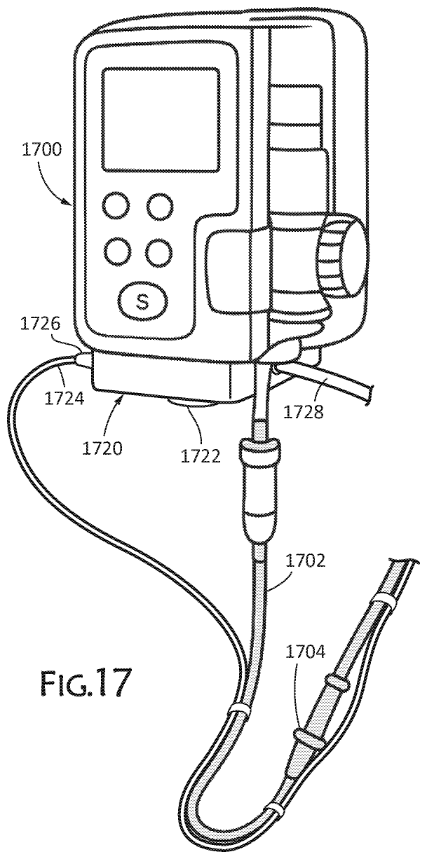

Referring now to FIGS. 16A-C, disclosed are various aspects of a fiber funnel cap 1602 that is designed to engage with the electronic illuminator, through the translucent ring or other receiving assembly on the housing to the lens of the electronic illuminator for transmitting line through the side scattering or side emitting fiber optic line. The fiber funnel cap, in one aspect, is also configured with a metal plate that may be utilized for magnetically locking the cap into the electronic illuminator or for tuning with a magnetic flux. For example, the metal plate 1604 may also be used for authentication and verification in coordination with the fiber detection assembly and the onboard hall sensor by a magnetic flux key or signature. In this regard, the metal plate 1604 is characterized by a specific angle vector relative to the onboard magnet assembly of the electronic illuminator, thus creating a magnetic flux signature or key.

Continuing, the fiber funnel cap 1602 may come in a variety of colors and the color is detected by the cap color detection assembly, wherein the MCU may power an LED driver to the specific cap color. Further, the fiber funnel cap 1602 may be equipped with bands, such as a multiple band code, or a SKU, or QR code, that allows for the R/G/B sensor of the cap color detection assembly to read the bands or code and transmit to the MCU a signal for the color to illuminate by the LED power driver. Therefore, the fiber optic cable, including the fiber funnel cap may be “programmed” from manufacture to illuminate a specific LED driver within the electronic illuminator. The system therefore reduces cognitive load on practitioners as the fiber funnel caps are coded with the matching color, reducing overhead and allowing design to provide function.

Continuing, FIG. 17 is a perspective view of an example medical infusion pump with an electronic illuminator module 1720 attached to the bottom and the corresponding side scattering fiber optic line 1724 attached to the medical fluid line of the medical infusion pump 1700. The primary driver of the medical infusion pump's operation is the mechanical delivery system, typically driven by stepper motors or linear actuators. These components control the movement of a syringe or the peristaltic compression of an IV tube to push fluid into the patient's bloodstream. The design ensures accurate flow rates ranging from milliliters per hour to micro-liters per hour, depending on the therapy type. Sensors are embedded within the system to detect flow irregularities, occlusions (blockages), air bubbles, and line disconnections, all of which are critical to preventing patient harm. These sensors may be coupled to the electronic illuminator module 1720 to cause patterns of light, or colors of light to illuminate on flow irregularities.

The electronic control system governs the interface between the mechanical components and the software logic on the medical infusion pump, and the electronic control system may be integrated over plug and play or data port to the MCU on the electronic illuminator module 1724. Further, the electronic illuminator module 1724 is comprised of a microcontroller or embedded processor that executes real-time commands from the activation of a on/off button 1722, which allows for rapid survey of the line by a nurse. The present disclosure contains minimum exterior surface parts/components, which reduce contamination, and reduces cognitive load, as a practitioner must only activate an on/off button 1722 to illuminate the side scattering fiber optic line 1724. Key hardware modules in the electronic illuminator module include power management circuits, memory storage for logs and configurations, communication interfaces (e.g., USB, Ethernet, Wi-Fi, Bluetooth), LED module(s), and cap detection sensors (proximity sensors).

In one aspect, the electronic illuminator module communicates with the medical infusion pump through a combined power and data connection. The power cable connecting the electronic illuminator module to the medical infusion pump is configured to carry both DC power for operating the module's components such as the MCU and other sensor assemblies configured to the PCB, and configured for bidirectional data signals for communication with the medical infusion pump's electronic control system.

In one embodiment, data transmission over the power line is achieved using a USB C interface, wherein power and data lines are configured. In another embodiment, the power connection utilizes a multi-conductor cable wherein dedicated conductors carry data signals using standard protocols such as RS-485, CAN bus, or USB, while separate conductors carry DC power. This configuration provides galvanic isolation between power and data paths.

The data transmitted from the electronic illuminator module to the medical infusion pump may include: (1) fiber optic cable detection status indicating whether a cable is properly connected; (2) cap color identification data for verifying correct medication line pairing; (3) LED operational status and fault conditions; (4) ambient light sensor readings; (5) authentication confirmation for genuine fiber optic cables; and (6) diagnostic or operational instructions from the medical infusion pump, including workflows or routines.

The data received by the electronic illuminator module from the medical infusion pump may include: (1) commands to illuminate specific LED colors independent of cap detection; (2) illumination patterns or flash sequences corresponding to pump alerts or alarms; (3) instructions to enter low-power standby mode; and (4) firmware update data.

In one aspect, the communication protocol implements a master-slave architecture wherein the medical infusion pump serves as the master device and the electronic illuminator module responds to polling requests. Message frames include header bytes, command codes, payload data, and cyclic redundancy check (‘CRC’) bytes for error detection.

In the example of FIG. 17, the medical infusion pump 1700 is configured with a medical infusion line 1702 for infusing medical fluids to a patient. The medical infusion line 1702 has a line impediment 1704. The side scattering fiber optic line 1724 is attached to the electronic illuminator module through the fiber funnel cap 1726 which is engaged to receive light from the LED inside the electronic illuminator module 1720.

The electronic illuminator module 1720 is disclosed with a power line 1728 to acquire power from the medical infusion pump 1700 or a wall outlet. The electronic illuminator module 1720 is shrouded in a protective housing, inside the protective housing is a printed circuit board (‘PCB’), a light emitting diode (‘LED’), a lens, and a power source. The power source, as stated may be directly drawing from a power port on the electronic illuminator or a wall outlet or a battery pack. The medical fluid line 1702 is coupled with clips or attachment points to the side scattering fiber optic cable 1724, therefore allowing the side scattering fiber optic cable 1724 to jump or hop over line impediments 1704.

In one aspect, the electronic illuminator module is comprised of at least one sensor assembly. The sensor assembly is comprised of a plurality of sensors, including an ambient light sensor, and a fiber detection assembly, both of which are measures that aid the user in determining the engagement of the side scattering fiber optic cable, as well as the illumination provided by the source of illumination, such as an LED. In another aspect, the electronic illuminator module may be comprised of an LED, an on/off switch, and a controller board, such as a PCB with a microcontroller unit. In further aspect, the electronic illuminator module may be comprised of a power supply, which converts the power or adjusts the power need, an LED, and an on/off switch. In other aspects, a sensor assembly may be applied, such as a proximity sensor, or an ambient light sensor.

Continuing, FIG. 18A is a perspective view of an example electronic illuminator module 1820. In the example, the electronic illuminator module 1820 has a protective housing and enclosed is the various components and sensor assemblies. In the example, power is routed to the MCU and PCB through the power cable 1828, such as a USB C data cable that allows for both power and data. In other examples, the power cable 1828 may only deliver power. The side scattering fiber optic cable 1824 is attached via the fiber funnel cap 1826 to the electronic illuminator module 1820 housing, that may comprise a translucent ring. Once connected the LED may be activated by the switch 1822 instructing the MCU to deliver power. The fiber funnel cap 1826 may also be configured to a translucent ring to allow for optical sensor assembly detection of ambient light. A practitioner may use the on/off switch 1822 or button to activate the LED through the MCU to transmit light through the lends down the side scattering fiber optic cable to aid in tracing the medical infusion fluid line to verify safety and compliance with the correct port into a patient.

Continuing, FIG. 18B is a diagram of an example electronic illuminator module with the housing removed. The diagram shows example components in the electronic illuminator module 1850, including the external power supply 1828 such as power from the medical infusion pump or wall outlet. Internal components comprise a power supply for converting the power and/or correcting voltage or amperage. The MCU/Radio also known as the microcontroller unit that provides the processing power to the LED driver, driving light from the 1826 LED. The ambient light sensor, and RGB sensor to determine and verify light is being transmitted, and the color of fiber funnel cap 1824 dictates the light to be transmitted. The MCU is further configured on the PCB and connected electronically to the other sensor assemblies and to the power supply.

The fiber detection assembly is one example of a sensor assembly and is incorporated to verify the fiber funnel cap is properly seated to receive the transmitted light from the LED, and to verify authenticity of the side scattering fiber optic line. The on/off switch 1822 or button is disclosed showing a direct connection to the logic of the power supply that is coupled to the external power 1828. The switch may further be configured to toggle certain options, such as a long press or a short press for certain operations—a long press, for example, may turn the device off, and a short press may cycle through different colors of LED illumination. Further, a series of presses may be configured, such as three short presses allows for pulses of LED light to be transmitted. Many other configurations may be added, including a second or third switch to allow for even further practitioner interaction, without significantly departing from the disclosure herein.

In one aspect, the electronic illuminator module 1850 is attached via adhesive strips to a medical infusion pump. In another aspect it is near or affixed to a wall that is near the medical infusion pump. In yet further aspects, the electronic illuminator module 1850 is affixed via physical fasteners through the housing and into the housing of the medical infusion pump.

### IV. Embodiments

Certain implementations of systems and methods consistent with the present disclosure are provided as follows:

Aspect 1. A system for medical infusion line illumination, comprising: (i) an electronic illuminator module configured to a medical infusion pump, the module comprising: a protective housing with an integrated activation switch, inside the protective housing is a microcontroller (‘MCU’), a light emitting diode (‘LED’), a lens, and a power source, wherein the power source is in electrical communication to the medical infusion pump to transmit both power and data to the MCU; and at least one sensor assembly. (ii) a side scattering fiber optic cable connected to the electronic illuminator module and traversing alongside a medical infusion line, the side scattering fiber optic cable comprising: a fiber funnel cap at a proximal end of the side scattering fiber optic cable to receive light from the LED through the lens; and a protective end cap at a distal end of the side scattering fiber optic cable, wherein an interior surface of the protective end cap is polished to reflect light back through the side scattering fiber optic cable.

Aspect 2. The system of aspect 1, wherein the at least one sensor assembly is an ambient light sensor, the ambient light sensor converts light intensity to a digital signal for the MCU to adjust the brightness of the LED.

Aspect 3. The system of aspect 2, wherein the light intensity is used by the MCU to turn off the LED based on a low light intensity indicating lights are off in a patient's room.

Aspect 4. The system of aspect 1, wherein the at least one sensor assembly is a fiber detection assembly having a three-dimensional magnetic flux density for authentication of the fiber funnel cap of the side scattering fiber optic cable.

Aspect 5. The system of aspect 4, wherein the fiber detection assembly comprises a hall effect sensor for authentication of the fiber funnel cap of the side scattering fiber optic cable.

Aspect 6. The system of aspect 4, wherein the fiber detection assembly is comprised of three magnets and a steel bar.

Aspect 7. The system of aspect 1, wherein the at least one sensor assembly is an optical power sensor assembly, the optical power sensor assembly detects travel time from a proximal end of the side scattering fiber optic cable to a distal end of the side scattering fiber optic cable.

Aspect 8. The system of aspect 1, wherein the at least one sensor assembly is a cap color detection assembly, the cap color detection assembly detects through a photo sensor a color of the fiber funnel cap of the side scattering fiber optic cable.

Aspect 9. The system of aspect 8, wherein the cap color detection assembly comprises a photoelectric sensor.

Aspect 10. The system of aspect 8, wherein the cap color detection assembly comprises a red green blue (‘RGB’) sensor.

Aspect 11. The system of aspect 8, wherein the cap color detection assembly comprises a multiple band code on the fiber funnel cap of the side scattering fiber optic cable, and an optical read on the illuminator module.

Aspect 12. The system of aspect 1, wherein the protective housing for the electronic illuminator module is comprised of an external heat sink and an attachment point of either double sided tape, adhesive, or mechanical fasteners that attach the electronic illuminator module to a wall of the medical infusion pump.

Aspect 13. A system for medical infusion line illumination, comprising: (i) an electronic illuminator module configured to a medical infusion pump, the module comprising: a protective housing with an integrated activation switch, inside the protective housing is a microcontroller (‘MCU’), a light emitting diode (‘LED’), a lens, and a power source; and at least one sensor assembly. (ii) a side scattering fiber optic cable connected to the electronic illuminator module and traversing alongside a medical infusion line, the side scattering fiber optic cable comprising: a fiber funnel cap at a proximal end of the side scattering fiber optic cable to receive light from the LED through the lens; and a protective end cap at a distal end of the side scattering fiber optic cable.

Aspect 14. The system of aspect 13, wherein the power source is the medical infusion pump or a wall outlet.

Aspect 15. The system of aspect 13, wherein the power source is the medical infusion pump and is further equipped to transmit data to and from the medical infusion pump.

Aspect 16. The system of aspect 13, wherein the electronic illuminator module further comprises a translucent cap, the translucent cap is utilized by an ambient light sensor to control the intensity of the LED.

Aspect 17. The system of aspect 13, wherein the at least one sensor assembly is an ambient light sensor, the ambient light sensor converts light intensity to a digital signal for the MCU to adjust the brightness of the LED.

Aspect 18. The system of aspect 13, wherein the at least one sensor assembly is a fiber detection assembly having a three-dimensional magnetic flux density for authentication of the fiber funnel cap of the side scattering fiber optic cable

Aspect 19. The system of aspect 13, wherein the protective end cap comprises a one-line antenna for signaling to the MCU to determine a distance of the side scattering fiber optic cable by signal strength received from a radio on the MCU.

Aspect 20. The system of aspect 13, wherein the at least one sensor assembly is a cap color detection assembly, the cap color detection assembly detects through a photo sensor a color of the fiber funnel cap of the side scattering fiber optic cable.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. A system for medical infusion line illumination, comprising:
(i) an electronic illuminator module configured to a medical infusion pump, comprising:
a protective housing with an integrated activation switch, inside the protective housing is a microcontroller (‘MCU’), a light emitting diode (‘LED’), a lens, and a power source, wherein the power source is in electrical communication to the medical infusion pump to transmit both power and data to the MCU; and
at least one sensor assembly.
(ii) a side scattering fiber optic cable connected to the electronic illuminator module and traversing alongside a medical infusion line, the side scattering fiber optic cable comprising:
a fiber funnel cap at a proximal end of the side scattering fiber optic cable to receive light from the LED through the lens; and
a protective end cap at a distal end of the side scattering fiber optic cable, wherein an interior surface of the protective end cap is polished to reflect light back through the side scattering fiber optic cable.

2. The system of claim 1, wherein the at least one sensor assembly is an ambient light sensor, the ambient light sensor converts light intensity to a digital signal for the MCU to adjust brightness of the LED.

3. The system of claim 2, wherein the light intensity is used by the MCU to turn off the LED based on a low light intensity indicating lights are off in a patient's room.

4. The system of claim 1, wherein the at least one sensor assembly is a fiber detection assembly having a three-dimensional magnetic flux density for authentication of the fiber funnel cap of the side scattering fiber optic cable.

5. The system of claim 4, wherein the fiber detection assembly comprises a hall effect sensor for authentication of the fiber funnel cap of the side scattering fiber optic cable.

6. The system of claim 4, wherein the fiber detection assembly is comprised of three magnets and a steel bar.

7. The system of claim 1, wherein the at least one sensor assembly is an optical power sensor assembly, the optical power sensor assembly detects travel time from the proximal end of the side scattering fiber optic cable to the distal end of the side scattering fiber optic cable.

8. The system of claim 1, wherein the at least one sensor assembly is a cap color detection assembly, the cap color detection assembly detects through a photo sensor a color of the fiber funnel cap of the side scattering fiber optic cable.

9. The system of claim 8, wherein the cap color detection assembly comprises a photoelectric sensor.

10. The system of claim 8, wherein the cap color detection assembly comprises a red green blue (‘RGB’) sensor.

11. The system of claim 8, wherein the cap color detection assembly comprises a multiple band code on the fiber funnel cap of the side scattering fiber optic cable, and an optical read on the illuminator module.

12. The system of claim 1, wherein the protective housing for the electronic illuminator module is comprised of an external heat sink and an attachment point of either double sided tape, adhesive, or mechanical fasteners that attach the electronic illuminator module to a wall of the medical infusion pump.

13. A system for medical infusion line illumination, comprising:
(i) an electronic illuminator module configured to a medical infusion pump, comprising:
a protective housing with an integrated activation switch, inside the protective housing is a microcontroller (‘MCU’), a light emitting diode (‘LED’), a lens, and a power source; and
at least one sensor assembly.
(ii) a side scattering fiber optic cable connected to the electronic illuminator module and traversing alongside a medical infusion line, the side scattering fiber optic cable comprising:
a fiber funnel cap at a proximal end of the side scattering fiber optic cable to receive light from the LED through the lens; and
a protective end cap at a distal end of the side scattering fiber optic cable.

14. The system of claim 13, wherein the power source is the medical infusion pump or a wall outlet.

15. The system of claim 13, wherein the power source is the medical infusion pump and is further equipped to transmit data to and from the medical infusion pump.

16. The system of claim 13, wherein the electronic illuminator module further comprises a translucent cap, the translucent cap is utilized by an ambient light sensor to control intensity of the LED.

17. The system of claim 13, wherein the at least one sensor assembly is an ambient light sensor, the ambient light sensor converts light intensity to a digital signal for the MCU to adjust brightness of the LED.

18. The system of claim 13, wherein the at least one sensor assembly is a fiber detection assembly having a three-dimensional magnetic flux density for authentication of the fiber funnel cap of the side scattering fiber optic cable.

19. The system of claim 13, wherein the protective end cap comprises a one-line antenna for signaling to the MCU to determine a distance of the side scattering fiber optic cable by signal strength received from a radio on the MCU.

20. The system of claim 13, wherein the at least one sensor assembly is a cap color detection assembly, the cap color detection assembly detects through a photo sensor a color of the fiber funnel cap of the side scattering fiber optic cable.
